(19) United States
(12) Patent Application Publication
(10) Pub. No.: US 2015/0175502 A1
(43) Pub. Date: Jun. 25, 2015

(54) METHOD FOR MAKING HIGH PURITY DICYCLOPENTADIENE

(71) Applicant: CPC Corporation, Taiwan, Taipei (TW)

(72) Inventors: Jyh-Haur HWANG, Chiayi City (TW); Wei-Bin SU, Chiayi City (TW); Chi-Yu CHEN, Chiayi City (TW); Karl Tze-Tang CHUANG, Chiayi City (TW); Cheng-Tsung HONG, Chiayi City (TW); Yung-Sheng HO, Chiayi City (TW)

(21) Appl. No.: 14/471,648

(22) Filed: Aug. 28, 2014

(30) Foreign Application Priority Data
Dec. 24, 2013 (TW) 102147913

(51) Int. Cl. C07C 2/50; C07C 4/22

(52) U.S. Cl. CPC C07C 2/50; C07C 4/22; C07C 2103/68

(57) ABSTRACT

A method for making high purity dicyclopentadiene comprises the following steps: providing a dimer feedstock comprising dicyclopentadiene and methyl dicyclopentadiene; blending the dimer feedstock and a diluent to obtain a first blend; cracking the first blend in a vapor phase at 300° C. to 400° C. to obtain a cracked feedstock; separating the cracked feedstock into a second blend and a third blend; dimerizing the second blend at 50° C. to 120° C. to obtain a fourth blend; and separating the fourth blend to obtain a recycled diluent and high purity dicyclopentadiene. By using saturated C5 hydrocarbon, saturated C6 hydrocarbon, benzene and the mixture thereof as the diluent and the steps mentioned above, the clogging problem of cracker tubes is solved and the cost of makeup diluent usage is lowered.

### BACKGROUND OF THE INVENTION

1. Field of the Invention

The present invention relates to a method for making high purity dicyclopentadiene (DCPD).

2. Description of the Prior Art(s)

Cyclopentadiene (CPD) dimerizes spontaneously to dicyclopentadiene and can reach thermodynamics equilibrium at room temperature. CPD tends to dimerize to DCPD at a temperature under 140° C. DCPD tends to crack into CPD at a temperature above 180° C. The production of DCPD is based on the principle described above. As CPD tends to dimerize to DCPD at a temperature lower than 140° C., DCPD, which is a hydrocarbon with ten carbons, is easy to separate from CPD, which is a hydrocarbon with five carbons. Or, low-purity DCPD is decomposed to CPD at a temperature higher than 180° C.; then, CPD is dimerized into high-purity DCPD at a temperature lower than 140° C.

U.S. Pat. No. 6,258,989 and U.S. Pat. No. 6,737,557 disclose a hydrocarbon feedstock originated from the bottom of a debutanizer is heated to let CPD dimerize into DCPD, thereby forming an effluent comprising DCPD; the effluent is distilled three times in sequence to obtain a DCPD stream comprising DCPD. However, the hydrocarbon feedstock originated from the bottom of the debutanizer comprises a small amount of methyl cyclopentadiene (MCPD), such that when CPD dimerizes, MCPD also dimerizes with CPD and itself. Thus, in addition to DCPD, the effluent comprises methyl dicyclopentadiene (MDCPD) and dimethyl dicyclopentadiene (DMDCPD). Without additional purification, those C11 and C12 hydrocarbons reduce the DCPD purity of the DCPD stream less than 90 wt %.

As for commercial DCPD production in C5-diene separation unit, a hydrocarbon feedstock rich in CPD, isoprene (IP) and 1,3-pentadiene (PD) is introduced to a CPD dimerization reactor and then to a distillation column to produce a DCPD stream comprising DCPD. However, in order to produce on specification IP and PD in the same process, a stream comprising an amount of hydrocarbons with ten or more carbons would flow back to that distillation column for producing DCPD, thereby reducing the purity of the DCPD product.

To increase the purity of DCPD, purification treatment is introduced to a low-purity DCPD stream. Typically, the purification treatment includes cracking, dimerizing, and fractionating steps for the low-purity DCPD stream. After purification treatment, a DCPD stream with DCPD having more than 95 wt % purity is produced.

Generally, the type of cracking step for DCPD purification is classified into liquid phase cracking and vapor phase cracking.

Liquid phase cracking of DCPD, as disclosed in U.S. Pat. No. 2,831,904, U.S. Pat. No. 3,590,089, U.S. Pat. No. 5,877,366 and U.S. Pat. No. 4,522,688, is that DCPD of a low-purity DCPD stream is decomposed into CPD at 200° C. to 300° C. with high boiling point inert liquids in a reactor. On the other hand, vapor phase cracking of DCPD, as disclosed in U.S. Pat. No. 5,321,177, U.S. Pat. No. 2,801,270 and U.S. Pat. No. 2,582,920, is that the method to crack the DCPD of a low-purity DCPD stream in a pyrolysis-type reactor at 300° C. to 400° C.

Since the temperature of liquid phase cracking is lower than that of vapor phase cracking, the former needs a longer residence time. Owing to the high reactivity of CPD, the long residence time and high temperature provide the conditions for the CPD and DCPD to form amounts of oligomer, which not only reduces the yield of CPD but also clogs the pipes and other process equipment. Relatively, the vapor phase cracking method has a higher yield of CPD for its shorter residence time in the cracker. But, the vapor phase cracking method also has the clogging problem, which is caused by the accumulation of the carbonized material and the deposit of polymerized DCPD on the surface of cracking tubes.

U.S. Pat. No. 5,321,177, U.S. Pat. No. 2,801,270 and U.S. Pat. No. 2,582,920 disclose methods to mitigate the clogging problem by introducing diluents, such as water, steam, inert hydrocarbon gases, phenylethene, coumarone and indene to vapor phase cracking.

However, those diluents mentioned above cannot sustain their diluting function in the downstream processing steps, namely fractionation and redimerization. In addition, those diluents mentioned above are not recycled for reuse; thus resulting in a significant increase in material costs.

### SUMMARY OF THE INVENTION

The main objective of the present invention is to introduce a suitable diluent to a method for making high purity dicyclopentadiene. The diluent can alleviate the clogging of cracker tubes in vapor phase cracking and increase the yield of DCPD and reduce the cost of dicyclopentadiene production.

The method for making high purity dicyclopentadiene comprises steps of:

providing a dimer feedstock comprising dicyclopentadiene and methyl dicyclopentadiene;

blending the dimer feedstock and a diluent selected from a group consisting of: a saturated C5 hydrocarbon, a saturated C6 hydrocarbon, benzene and a mixture thereof, to obtain a first blend;

cracking the first blend in a vapor phase at a temperature ranging from 300° C. to 400° C. to obtain a cracked feedstock comprising cyclopentadiene, methyl cyclopentadiene and the diluent;

separating the cracked feedstock into a second blend and a third blend, the second blend comprising cyclopentadiene and the diluent, and the third blend comprising methyl cyclopentadiene and the diluent;

heating the second blend to a dimerizing temperature ranging from 50° C. to 120° C. to obtain a fourth blend comprising dicyclopentadiene and the diluent; and

separating the fourth blend to obtain a recycled diluent and a high purity dicyclopentadiene;

wherein the diluent comprises the recycled diluent.

The “high purity dicyclopentadiene” in accordance with the present invention is designated to a material having a concentration of dicyclopentadiene greater than 95 weight percent (wt %).

The “diluent” in accordance with the present invention is designated as a material being chemically inert and completely miscible to dicyclopentadiene and methyl dicyclopentadiene. The diluents which meet the above requirements include C5 and C6 hydrocarbons. Preferably, the saturated C5 hydrocarbon is selected from a group consisting of: cyclopentane, n-pentane, and isopentane.

Preferably, the saturated C6 hydrocarbon is selected from a group consisting of: cyclohexane, an isomer of cyclohexane, n-hexane and an isomer of n-hexane. The isomer of n-hexane comprises 2-methyl pentane. The isomer of cyclohexane comprises methyl cyclopentane.

Preferably, the boiling points of the saturated C5 hydrocarbon and saturated C6 hydrocarbon are between the boiling point of cyclopentadiene and the boiling point of methyl cyclopentadiene.

By using the saturated C5 hydrocarbon, saturated C6 hydrocarbon, benzene or the mixture thereof as the diluent to dilute the dimer feedstock, the first blend with a lower concentration of DCPD and MDCPD than the dimer feedstock is obtained. By introducing the first blend, instead of the dimer feedstock, to a cracker for vapor phase cracking, the clogging problem of cracker tubes is solved.

As the cracked feedstock is separated, the reaction loss of cyclopentadiene is reduced due to the presence of diluent in the separation process. Besides, the diluent is recyclable and reusable in the present invention. Therefore, the cost of using the diluent is lowered.

Since the dimerization of CPD is an exothermic reaction, the presence of diluent in the second blend reduces both the reaction rate of CPD dimerization and the amount of heat created in a unit time. This can prevent the excessive rise of temperature in the adiabatic dimerization reactor. It is known that high temperature inside the CPD dimerization reactor incurs more trimerization of CPD, which reduces the purity of DCPD product.

Preferably, the method in accordance with the present invention further comprises a step of:

separating the recycled diluent into a first recycled diluent and a second recycled diluent;

the step of blending the dimer feedstock and the diluent to obtain the first blend comprises:

blending the dimer feedstock and the first recycled diluent to obtain the first blend; and

the step of cracking the first blend in the vapor phase at the cracking temperature ranging from 300° C. to 400° C. to obtain the cracked feedstock comprises:

cracking the first blend in the vapor phase at the cracking temperature ranging from 300° C. to 400° C. into an intermediate product; and

blending the intermediate product with the second recycled diluent to obtain the cracked feedstock.

Preferably, the method in accordance with the present invention further comprises a step of:

separating the recycled diluent into a first recycled diluent and a third recycled diluent;

the step of blending the dimer feedstock and the diluent to obtain the first blend comprises:

blending the dimer feedstock and the first recycled diluent to obtain the first blend; and

the step of heating the second blend to the dimerizing temperature ranging from 50° C. to 120° C. to obtain the fourth blend comprises:

blending the second blend and the third recycled diluent and then heating to the dimerizing temperature ranging from 50° C. to 120° C. to obtain the fourth blend.

More preferably, the step of separating the recycled diluent into the first recycled diluent and the second recycled diluent comprises:

separating the recycled diluent into a first recycled diluent, a second recycled diluent, and a third recycled diluent; and

the step of heating the second blend to the dimerizing temperature ranging from 50° C. to 120° C. to obtain the fourth blend comprises:

blending the second blend and the third recycled diluent and then heating to the dimerizing temperature ranging from 50° C. to 120° C. to obtain the fourth blend.

Based on the above, the recycled diluent in accordance with the present invention is not only to reduce the concentration of dimers in dimer feedstock for the vapor phase cracker but also to lower the concentration of CPD in the next distillation and redimerization processes. Thus, the reaction loss of cyclopentadiene is reduced and the yield of the high purity dicyclopentadiene is increased.

Preferably, the method in accordance with the present invention further comprises steps of:

heating the third blend to dimerize the methyl cyclopentadiene of the third blend and obtain a high-boiling point hydrocarbon feedstock; and

separating the high-boiling point hydrocarbon feedstock to obtain a high-boiling point hydrocarbon and a fourth recycled diluent; and

the step of blending the dimer feedstock and the diluent to obtain the first blend comprises:

blending the dimer feedstock and the fourth recycled diluent to obtain the first blend.

More preferably, the method in accordance with the present invention further comprises the steps of:

heating the third blend to dimerize the methyl cyclopentadiene of the third blend and obtain the high-boiling point hydrocarbon feedstock; and

separating the high-boiling point hydrocarbon feedstock to obtain the high-boiling point hydrocarbon and the fourth recycled diluent; and

the step of blending the dimer feedstock and the diluent to obtain the first blend comprises:

blending the dimer feedstock, the first recycled diluent, and the fourth recycled diluent to obtain the first blend.

The diluent in accordance with the present invention is recycled from the third blend, which is the by-product of the method; thereby the cost of using the diluent is lowered.

Preferably, the step of providing the dimer feedstock comprises:

separating the hydrocarbon stream originated from the bottom of a debutanizer into a hydrocarbon feedstock comprising hydrocarbons with five carbons and another hydrocarbon feedstock comprising hydrocarbons with six or more carbons; and

separating the hydrocarbon feedstock comprising hydrocarbons with six or more carbons to obtain the dimer feedstock.

More preferably, the hydrocarbon stream originated from the bottom of debutanizer comprises a concentration of dicyclopentadiene ranging from 5 wt % to 20 wt % and a concentration of methyl dicyclopentadiene ranging from 0.5 wt % to 5 wt %.

In accordance with the present invention, the dimer feedstock is, but not limit to, recycled from the hydrocarbon stream originated from the bottom of a debutanizer.

Preferably, the step of blending the dimer feedstock and the diluent to obtain a first blend comprises:

blending the dimer feedstock and the diluent comprising a makeup diluent selected from a group consisting of: saturated C5 hydrocarbon, saturated C6 hydrocarbon, benzene and a mixture thereof to obtain the first blend.

Based on the above, by adding the makeup diluent, the total amount of the diluent can be kept sufficient in the present invention.

In accordance with the present invention, the “makeup diluent” is designated as a substance being inert and completely miscible to dicyclopentadiene and methyl dicyclopentadiene, such as saturated C5 hydrocarbon, saturated C6 hydrocarbon, benzene and a mixture thereof.

Preferably, the dimer feedstock comprises a concentration of dicyclopentadiene and methyl dicyclopentadiene ranging from 50 wt % to 85 wt %.

Preferably, the first blend comprises a total concentration of dicyclopentadiene and methyl dicyclopentadiene ranging from 10 wt % to 50 wt %.

In accordance with the present invention, the “C4 hydrocarbon, “C5 hydrocarbon”, “C6 hydrocarbon”, “C7 hydrocarbon”, “C8 hydrocarbon”, “C9 hydrocarbon”, “C10 hydrocarbon”, “C11 hydrocarbon” and “C12 hydrocarbon” are respectively designated as a hydrocarbon with four, five, six, seven, eight, nine, ten, eleven and twelve carbons.

Other objectives, advantages, and novel features of the present invention will become more apparent from the following detailed description when taken in conjunction with the accompanying drawings.

### BRIEF DESCRIPTION OF THE DRAWINGS

FIG. 1 is a schematic view of a system of a method for making a high purity dicyclopentadiene in accordance with the present invention;

FIG. 2 is another schematic view of a system of a method for making a high purity dicyclopentadiene in accordance with the present invention.

### DETAILED DESCRIPTION OF THE PREFERRED EMBODIMENTS

In the preferred embodiments of the present invention, a hydrocarbon stream originated from the bottom of a debutanizer is taken as the raw material for obtaining a dimer feedstock comprising the CPD dimers, which are DCPD and MDCPD. Then a recyclable diluent is applied. A vapor phase cracking is performed to crack CPD dimers to obtain CPD monomer. Afterwards, a distillation process is used to isolate cyclopentadiene. Then cyclopentadiene is dimerized to dicyclopentadiene. In the end, a distillation is performed to isolate dicyclopentadiene, and a high purity dicyclopentadiene comprising a concentration of dicyclopentadiene over 95 wt % is obtained.

In a preferred embodiment of the present invention, the raw material of the method for making the high purity dicyclopentadiene is originated from a by-product of cracking ethane, propane, naphtha, or gas oil, which is generated from the bottom of a debutanizer. A typical composition of this stream is summarized in Table 1, shown below:

TABLE 1
Composition of an oil originated from the bottom of a debutanizer

| Composition | Concentration (wt %) |
|---|---|
| C4 hydrocarbons | 1.82 |
| CPD | 9.67 |
| Other C5 hydrocarbons | 22.71 |
| MCPD | 1.1 |
| Other C6 hydrocarbons | 44.56 |
| C7 hydrocarbons | 5.01 |
| C8 hydrocarbons | 0.25 |
| C9 hydrocarbons | 0.38 |
| DCPD | 7.97 |
| Other C10 hydrocarbons | 0.82 |
| MDCPD | 2.15 |
| Other C11 hydrocarbons | 0.73 |
| DMDCPD | 0.14 |
| Other hydrocarbons with | 2.68 |
| 12 and more carbons | |

### Embodiment 1

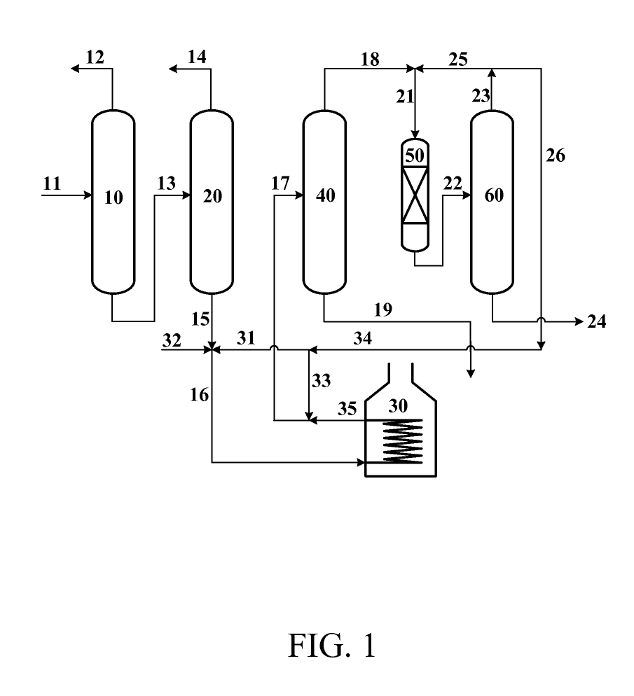

With reference to FIG. 1, a method for making high purity dicyclopentadiene of the present embodiment is as follows.

A hydrocarbon stream originated from the bottom of a debutanizer is fed to a first distillation column 10 by line 11 to remove C5 and lighter hydrocarbons by the overhead stream withdrawn via line 12. The bottom stream of the first distillation column 10 discharged via line 13 is introduced to the 14^{th} plate of second distillation column 20 via line 13 to remove the C6 to C8 hydrocarbons by the overhead stream of column 20 via line 14. The bottom stream of second distillation column 20 that is withdrawn via line 15 is a dimer feedstock which contains concentrated dicyclopentadiene and methyl dicyclopentadiene.

The overhead stream withdrawn via line 12 is named as a hydrocarbon feedstock comprising hydrocarbons with five carbons. The bottom stream of first distillation column 10 discharged via line 13 is named as a hydrocarbon feedstock comprising hydrocarbons with six or more carbons. The overhead stream of column 20 via line 14 is named as an aromatic hydrocarbon feedstock.

By adding a recycled diluent via line 31 and a makeup diluent via line 32 to the dimer feedstock in line 15, a combined stream of the recycled diluent via line 31 and the dimer feedstock in line 15 formed in line 16 is the first blend. In addition, the diluent is selected from a group consisting of: a saturated C5 hydrocarbon, a saturated C6 hydrocarbon, benzene, and a mixture thereof. Preferably, the concentration of DCPD and MDCPD in the first blend ranges from 10 wt % to 50 wt % and more preferably, ranges from 10 wt % to 30 wt %.

The first blend in line 16 is charged to a cracker 30 to perform a vapor phase cracking of CPD dimers and obtain an intermediate product. The intermediate product is then mixed with a second recycled diluent coming from line 33 to obtain a cracked feedstock mainly comprising CPD, MCPD and the diluent. Preferably, the pressure for the vapor phase cracking is between 0 kg/cm² G and 10 kg/cm² G, and more preferably, the cracking pressure is between 0 kg/cm² G and 3.5 kg/cm² G. A preferred temperature for the vapor phase cracking to decompose CPD dimers ranges from 300° C. to 400° C., and more preferably, ranges from 320° C. to 360° C.

The cracked feedstock is introduced to a heavy removal column 40 via lines 35 and 17, to recover CPD and diluent in the cracked feedstock by the overhead stream of the heavy removal column 40 via line 18, and to discharge MCPD and the remaining hydrocarbons of the cracked feedstock by the bottom stream of the heavy removal column 40 via 19. The overhead stream and the bottom stream of the heavy removal column 40 are named as a second blend and a third blend respectively. Preferably, the top pressure of heavy removal column 40 is between −0.6 kg/cm² G and 2.0 kg/cm² G, and more preferably, is between −0.5 kg/cm² G and 0.5 kg/cm² G.

After mixing the second blend in line 18 with a third recycled diluent in line 25, the combined stream of the second blend in line 18 and the third recycled diluent in line 25 formed in line 21 is fed into a first dimerization reactor 50 to let CPD dimerize and the effluent of the first dimerization reactor 50 is a fourth blend comprising dicyclopentadiene and the diluent. The fourth blend is discharged via line 22. Preferably, the pressure for the first dimerization reactor 50 ranges from 1 kg/cm² G to 20 kg/cm² G, and more preferably, ranges from 9 kg/cm² G to 13 kg/cm² G. Preferably, an inlet (line 21) temperature of the first dimerization reactor 50 ranges from 60° C. to 120° C., and more preferably, ranges from 80° C. to 110° C. Preferably, an outlet (line 22) temperature of the first dimerization reactor 50 is between 110° C. and 150° C., and more preferably, is between 115° C. and 130° C. Preferably, the temperature of the first dimerization reactor 50 ranges from 50° C. to 120° C., and more preferably, ranges from 80° C. to 120° C.

The fourth blend is fed into a first recovery column 60 by line 22 to recover the diluent in the fourth blend by the overhead stream of the first recovery column 60 via line 23. The bottom stream of the first recovery column 60 via line 24 is a high purity dicyclopentadiene comprising a concentration of DCPD more than 95 wt %. A preferred pressure for the first recovery column 60 ranges from −0.8 kg/cm² G to 2.0 kg/cm² G, and more preferably, ranges from −0.7 kg/cm² G to −0.4 kg/cm² G.

The recycled diluent in line 23 is divided into three parts: a first recycled diluent piped via lines 26, 34, and 31; a second recycled diluent piped via lines 26, 34, and 33; and a third recycled diluent piped via line 25.

In the present embodiment, when cyclopentane, whose boiling point is close to that of CPD, is selected as the diluent, almost all the diluent in the cracked feedstock is recovered with CPD from the overhead stream of a distillation column. After the step of dimerization to convert CPD into DCPD, the diluent eventually will be separated from DCPD by a distillation and then be recycled.

### Embodiment 2

The differences between the present embodiment and Embodiment 1 are as follows.

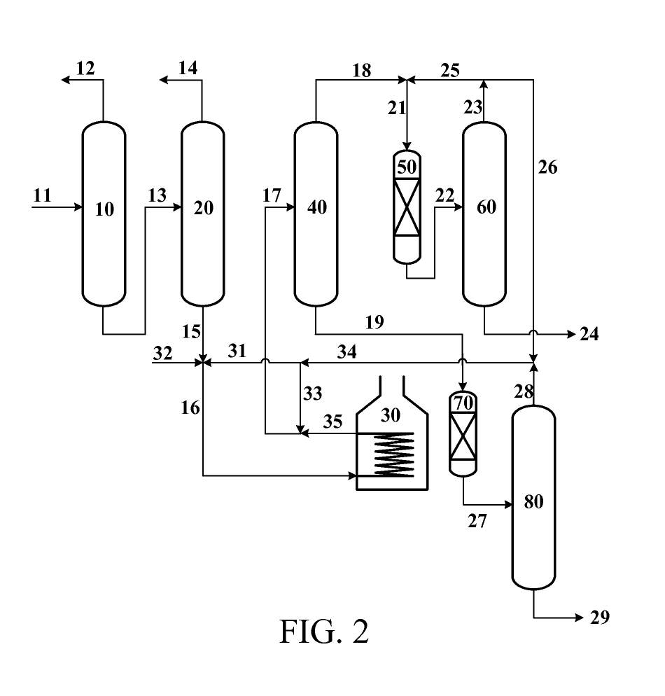

Referring to FIG. 2, the third blend is fed into a second dimerization reactor 70 via line 19 and the effluent of reactor 70, which is a high-boiling point hydrocarbon feedstock, is discharged from line 27. A preferred pressure for the second dimerization reactor 70 ranges from 1 kg/cm² G to 20 kg/cm² G, and more preferably, ranges from 5 kg/cm² G to 10 kg/cm² G. Preferably, an inlet (line 19) temperature of reactor 70 is between 130° C. and 180° C., and more preferably, is between 150° C. and 170° C. Preferably, an outlet (line 27) temperature of the second dimerization reactor 70 is between 150° C. and 190° C., and more preferably, is between 150° C. and 170° C. Preferably, the temperature for the second dimerization reactor 70 ranges from 130° C. to 200° C., and more preferably, ranges from 170° C. to 180° C.

The high boiling-point hydrocarbon feedstock is introduced into a second recovery column 80 via line 27 to recover the diluent in the high boiling-point hydrocarbon feedstock by the overhead stream of the second recovery column 80 via line 28, which is a fourth recycled diluent, and to withdraw the remaining hydrocarbons of the high boiling-point hydrocarbon feedstock from the bottom stream of the second recovery column 80 via line 29, as a fuel. The fourth recycled diluent is piped by lines 34 and 31. Preferably, the top pressure for column 80 ranges from −0.8 kg/cm² G to 2.0 kg/cm² G, and more preferably, ranges from −0.2 kg/cm² G to 0.4 kg/cm² G.

The dimer feedstock is blended with the diluent and the fourth recycled diluent coming from line 31.

The method for making high purity dicyclopentadiene in accordance with the present invention is described in detail as above. The technical means of the present invention is further elaborated with the following embodiments.

A simulation model has been built in the commercial process simulator, ASPEN 14. The thermodynamics property method of this simulation model is UNIQUAC equation (UNIversal QUAsi-Chemical equation), where the built-in binary component parameters are applied and missing binary parameters are predicted by UNIFAC model (UNIQUAC Functional-group Activity Coefficients).

In addition to the dimerization of CPD, dimerization of MCPD and the codimerization of MCPD and CPD, the following reactions are also considered in the simulation examples: CPD codimerizes with isoprene to form cyclopentadiene-isoprene dimer (CPDIP), CPD codimerizes with piperylene to form cyclopentadiene-piperylene dimer (CPDPD), and the trimerization of CPD forms tricyclopentadiene (TCPD). Those chemical reactions are listed in the following.

2 CPD→DCPD

2 MCPD→DMDCPD

CPD+MCPD→MDCPD

CPD+IP→CPDIP

CPD+PD→CPDPD

CPD+DCPD→TCPD

The kinetic parameters of the reactions mentioned above are taken from laboratory experiments and literatures. (ref: J. Krupka, Petroleum & Coal. 52(4), 290(2010), E. V. Nurullina et al., Russian J. Appl. Chem. 74(9), 1590(2001), I. Palmova et al., Chem. Eng. Sci. 56, 927(2001), W. C. Herndon et al., J. Org. Chem. 32(3), 526(1967) and W. B. Su et al., J. Petroleum 45(2), 63(2009)).

With reference to FIGS. 1 and 2, numbers of the theoretical plates for each distillation column are given as follows. The first distillation column 10 has 36 theoretical plates. The second distillation column 20 has 28 theoretical plates. The heavy removal column 40 has 16 theoretical plates. The first recovery column 60 has 12 theoretical plates. The second recovery column 80 has 19 theoretical plates. The plate number is counted from the top to bottom. The first plate is the condenser and the last plate is the reboiler. The tray spacing is set at 0.61 meters (m). Each distillation column is simulated under reactive distillation mode.

The setting of the reactive distillation mode is given as follows. Since the temperature of the first plate is lower than 55° C., the first plate is assumed to have no chemical reaction. The setting of the rest plates is that chemical reactions such as dimerization of dienes, codimerization of dienes and cracking of dimers occur in liquid phase.

The liquid volume of the bottom reboiler determines the residence time of the liquid, which is preset as 10 minutes. Except the last plate, the liquid holdup on each plate is 10 volume percent (vol. %) of tray spacing. The diameter of each distillation column is determined by the operating parameters, such as column pressure, reflux ratio, and distillate-to-feed mass ratio (D/F).

The diameter of each distillation column is determined with a built-in formula of ASPEN 14 and then the liquid holdup on each plate is calculated, according to the volume for dienes to undergo dimerization.

### EXAMPLE 1

### Preparation of Dimer Feedstock Comprising DCPD and MDCPD

A hydrocarbon stream originated from the bottom of a debutanizer was fed into a batch distillation apparatus with 15 theoretical plates. The experimental runs were carried out under ambient pressure at the top and the overhead reflux ratio was of 5.0. After the initial charge and system installation completed, the kettle started to heat up. When the temperature of bottom reached 155° C., the heat input to the distillation column was shut down. As the system cooled down, the bottom liquid of batch distillation was a dimer feedstock. The recovery of bottom liquid relative to feed was about 25%. The feed and bottom compositions of batch distillation were shown in Table 2.

TABLE 2
Feed and bottom compositions of the
batch distillation for Example 1

| | Feed | Bottom |
|---|---|---|
| Comp. | Conc. (wt %) | Conc. (wt %) |
| C4 hydrocarbons | 1.68 | 0.00 |
| CPD | 5.25 | 0.45 |
| Other C5 hydrocarbons | 24.00 | 0.01 |
| MCPD | 0.85 | 0.12 |
| Other C6 hydrocarbons | 42.26 | 2.02 |
| C7 hydrocarbons | 6.69 | 14.58 |
| C8 hydrocarbons | 0.61 | 2.32 |
| C9 hydrocarbons | 0.31 | 2.00 |
| DCPD | 10.64 | 44.94 |
| Other C10 hydrocarbons | 1.64 | 6.86 |
| MDCPD | 3.47 | 13.59 |
| Other hydrocarbons with | 2.59 | 13.11 |
| 11 and more carbons | | |

As shown in Table 2, the concentrations of DCPD and MDCPD were increased fourfold by batch distillation and both components showed no sign of decomposition at 155° C.

### EXAMPLE 2

### Cracking of a First Blend Comprising a Dimer Feedstock and a Diluent

A first blend containing 50% by weight of diluent and 50% by weight of the dimer feedstock obtained in Example 1 was decomposed under an average cracking temperature of 298° C. to obtain a cracked gas. The residence time of the first blend in the tube of the cracker was 0.5 seconds. The cracked gas was totally condensed by chilled water of 0° C. The outlet of the cracker was under ambient pressure.

The experimental procedures mentioned above were repeated, except that the average cracking temperature changed to 345° C., to obtain additional results.

The compositions of the two condensed liquids obtained under two different cracking temperatures were analyzed by a gas chromatography and shown in Table 3.

In the present example, the diluent was n-hexane (manufactured by Merck with purity higher than 99%). The experimental runs were conducted in a cracker made from a stainless tube with diameter of ⅛″ and a length of 195.5 centimeters. The cracker tube was embedded in a spiral trench on the surface of a metal bar, which was vertically mounted in a thermal insulated box. The metal bar was heated by three electric heating rods, which were inserted in vertical holes drilled in the bar. Heating power of the metal bar was adjusted by two temperature control loops with two temperature indicators and total power was up to 3,000 watts.

With reference to Table 3, a conclusion was made that the diluent (n-hexane) in the feed did not decompose in the cracker at temperature up to 345° C.

TABLE 3
Compositions of the two condensed liquids in Example 2

Cracking temp.

| | 298° C. | 345° C. |
|---|---|---|
| Comp. | Conc. (wt %) | Conc. (wt %) |
| C4 hydrocarbons | 0.01 | 0.06 |
| CPD | 12.61 | 26.35 |
| Other C5 hydrocarbons | 0.06 | 0.11 |
| n-hexane | 50.54 | 51.03 |
| MCPD | 2.42 | 5.03 |
| Other C6 hydrocarbons | 1.40 | 1.67 |
| C7 hydrocarbons | 7.88 | 7.50 |
| C8 hydrocarbons | 1.33 | 1.01 |
| C9 hydrocarbons | 1.15 | 1.09 |
| DCPD | 11.78 | 0.27 |
| Other C10 hydrocarbons | 2.14 | 2.21 |
| MDCPD | 4.06 | 0.56 |
| Other hydrocarbons with | 4.61 | 3.10 |
| 11 and more carbons | | |

### EXAMPLE 3

### Thermal Stability of Saturated C6 Hydrocarbons and Benzene

The part of results in Example 2 has proven that n-hexane was substantially non-crackable under a temperature up to 345° C. In the present example the injection temperature of a gas chromatography (GC) was set for a temperature higher than 300° C. to simulate the condition of a high temperature cracker.

To verify other types of C6 saturated and aromatic hydrocarbons are also non-crackable under the cracker condition, the mixture shown in Table 4 below was injected into Agilent 6890 GC at the cracker operating temperature of 350° C. Demonstrated in Table 4, the results again show none of these hydrocarbons are non-crackable.

TABLE 4
Compositions of the C6 and C7 hydrocarbons-rich
sample measured by a gas chromatography with two
different injection temperatures in Example 3

| | Injection temperature | | |
|---|---|---|---|
| | 160° C. | 350° C. | Conc. Variation |
| | Conc. | Conc. | Conc.(350° C.)/ |
| Comp. | (wt %) | (wt %) | Conc.(160° C.) × 100% |
| Unbranched | 5.613 | 5.599 | 99.8% |
| alkane with 6 | | | |
| carbons | | | |
| Isoalkane with 6 | 1.866 | 1.859 | 99.6% |
| carbons | | | |
| Isoalkene with 6 | 6.816 | 6.89 | 101.1% |
| carbons | | | |
| Cycloalkane with | 6.193 | 6.203 | 100.2% |
| 6 carbons | | | |
| Benzene | 72.400 | 72.342 | 99.9% |

### EXAMPLE 4

### Preparation of a Dimer Feedstock

Referring to FIG. 1, a hydrocarbon stream from the bottom of a debutanizer, composition given in Table 1, is fed to the 18^{th} plate of the first distillation column 10 via line 11 at rate of 42,000 kg/hr, to remove C5 and lighter hydrocarbons. The bottom of column 10 is introduced to the 14^{th} plate of second distillation column 20 via line 13 at temperature 121.3° C. to remove the C6 to C8 hydrocarbons from the overhead stream of column 20 via line 14. The bottom stream of column 20 with concentrated DCPD and MDCPD at rate of 6,805 kg/hr is withdrawn through line 15 at temperature 156.9° C. The bottom stream of column 20 is a dimer feedstock. The first distillation column 10 is operated under a top pressure of 1.0 kg/cm² G, a reflux ratio of 3.0, and a distillate-to-feed mass ratio (D/F) of 0.366. The second distillation column 20 is operated under a top pressure of −0.7336 kg/cm² G, a reflux ratio of 2.0, and a distillate-to-feed mass ratio (D/F) of 0.744. Since dimerization and decomposition reactions occur in both columns, operations of column 10 and column 20 are simulated with Aspen Plus under reactive distillation mode.

Composition of dimer feedstock withdrawn from the bottom of column 20 is summarized in Table 5.

TABLE 5
Composition of the dimer feedstock in Example 4

| Comp. | Conc. (wt %) |
|---|---|
| CPD | 0.01 |
| C7 hydrocarbons | 1.68 |
| C8 hydrocarbons | 0.80 |
| C9 hydrocarbons | 2.36 |
| DCPD | 52.29 |
| Other C10 hydrocarbons | 5.07 |
| MDCPD | 13.45 |
| Other hydrocarbons with | 24.33 |
| 11 and more carbons | |

### EXAMPLE 5

### Selection of a Diluent

As mentioned previously, the purpose of adding diluent via lines 31 (recycled) and 32 (makeup) to the dimer feed in line 15 in FIG. 1, is to reduce its concentration of CPD dimers from approximately 60 wt % to 30 wt % in line 16, which is the first blend. The diluted dimer feedstock is then fed to the cracker 30. Cracker 30 is simulated using the tubular reactor model in ASPEN 14 operated under isothermal mode at 340° C. The volume of the cracker tube is set at 0.3989 cubic meters (m³) and the residence time is within the range of 0.5 to 1.0 second. The effluent of cracker 30 is introduced to the 11^{th} plate of the heavy removal column 40 via line 17, to recover CPD and the most portion of diluent by the overhead stream of column 40 via line 18, and to remove the remaining hydrocarbons of the feed in line 17 by the bottom stream of column 40 via line 19. The overhead stream and the bottom stream of column 40 are a second blend and a third blend respectively.

The present example demonstrates not only the operation of the cracker 30, but also illustrates the influence of the diluent in the operation of the column 40 crucial to the recovery and the purity of CPD produced in the overhead of the column. Based on the boiling point of CPD and MCPD, seven different diluents are selected for Aspen Plus simulation, including n-pentane, cyclopentane, 2-methyl pentane, n-hexane, methyl cyclopentane, benzene, and toluene.

In order to obtain a recovered CPD product with high purity in the overhead of column 40, the maximum allowable MCPD in the overhead stream (line 18) is 1% of the feed to column 40, while the maximum loss of CPD in the bottom stream (line 19) of column 40 is 0.15% of the feed to the column 40. In simulation calculations, reflux ratio (RR) and distillate-to-feed mass ratio (D/F) of column 40 are adjusted to meet these two requirements. Column 40 is simulated under reactive distillation mode, the top pressure of column 40 is 0.5 kg/cm² G, and the feed enters column 40 on the 11^{th} plate of column 40.

The flow rates and compositions of the first blend with different diluents and their cracking results are given in Tables 6 and 7.

TABLE 6
Flow rates and compositions of the inlet (line 16) and outlet (line 17) of cracker 30 in Example 5

| | Diluent | | | | | | | |
|---|---|---|---|---|---|---|---|---|
| | n-pentane | | cyclopentane | | 2-methyl pentane | | n-hexane | |
| Line No. | 16 | 17 | 16 | 17 | 16 | 17 | 16 | 17 |
| Flow rate (kg/hr) | 15642 | 15642 | 15642 | 15642 | 15642 | 15642 | 15642 | 15642 |
| CPD (wt %) | 2.24 | 27.64 | 1.82 | 27.22 | 1.73 | 27.13 | 0.47 | 25.87 |
| MCPD (wt %) | 0.00 | 4.60 | 0.01 | 4.61 | 0.04 | 4.64 | 0.16 | 4.76 |
| Diluent (wt %) | 54.25 | 54.25 | 54.67 | 54.67 | 54.71 | 54.71 | 55.85 | 55.85 |
| DCPD (wt %) | 22.75 | 0.00 | 22.75 | 0.00 | 22.75 | 0.00 | 22.75 | 0.00 |
| MDCPD (wt %) | 5.85 | 0.00 | 5.85 | 0.00 | 5.85 | 0.00 | 5.85 | 0.00 |
| Other (wt %) | 14.90 | 13.50 | 14.90 | 13.50 | 14.92 | 13.52 | 14.9 | 13.52 |

TABLE 7
Flow rates and compositions of the inlet (line 16)
and outlet (line 17) of cracker 30 in Example 5

| | Diluent | | | | | |
|---|---|---|---|---|---|---|
| | methyl cyclopentane | | Benzene | | toluene | |
| Line No. | 16 | 17 | 16 | 17 | 16 | 17 |
| Flow rate (kg/hr) | 15642 | 15642 | 15642 | 15642 | 15642 | 15642 |
| CPD (wt %) | 0.71 | 26.10 | 0.60 | 26.00 | 1.02 | 26.42 |
| MCPD (wt %) | 0.14 | 4.74 | 0.11 | 4.71 | 0.12 | 4.72 |
| Diluent (wt %) | 55.63 | 55.63 | 55.77 | 55.77 | 55.84 | 55.84 |
| DCPD (wt %) | 22.75 | 0.00 | 22.75 | 0.00 | 22.75 | 0.00 |
| MDCPD (wt %) | 5.85 | 0.00 | 5.85 | 0.00 | 5.85 | 0.00 |
| Other (wt %) | 14.92 | 13.52 | 14.92 | 13.52 | 14.42 | 13.02 |

The simulated operating parameters, calculated recovery of diluent as well as calculated loss and recovery of CPD in the overhead of column 40 under different diluents are shown in Tables 8A and 8B. The flow rates and compositions of the overhead in line 18 and the bottom in line 19 for column 40 are shown in Tables 9A and 9B.

TABLE 8A
Operation parameters and results of
heavy removal column 40 in Example 5

| | Diluent | | | |
|---|---|---|---|---|
| | | Cyclo- | 2-methyl | |
| | n-pentane | pentane | pentane | n-hexane |
| Boiling point of | 36.1 | 49.3 | 60.3 | 68.7 |
| diluent (° C.) | | | | |
| Top pressure (kg/cm²G) | 0.5 | 0.5 | 0.5 | 0.5 |
| Condensation | 46.0 | 57.4 | 61.4 | 56.3 |
| Temperature (° C.) | | | | |
| D/F | 0.808 | 0.802 | 0.678 | 0.299 |
| Reflux ratio | 0.36 | 1.30 | 5.40 | 9.99 |
| Bottom temperature | 187.9 | 158.7 | 97.0 | 93.1 |
| (° C.) | | | | |
| Temperatures from 2^{nd} | 48.2-122.4 | 60.1-85.6 | 66.6-79.6 | 60.5-87.6 |
| to 15^{th} plates(° C.) | | | | |
| Column diameter (m) | 1.16 | 1.52 | 2.44 | 2.23 |
| CPD reaction loss in | −169 | −149 | −96 | −327 |
| column 40 (kg/hr) | | | | |
| CPD recovery at the | 95.94 | 96.41 | 97.59 | 91.75 |
| overhead stream (%) | | | | |
| Diluent recovery at the | 99.96 | 98.69 | 75.34 | 10.84 |
| overhead stream (%) | | | | |

TABLE 8B
Operation parameters and results of
heavy removal column 40 in Example 5

| | Diluent | | |
|---|---|---|---|
| | methyl | | |
| | cyclo- | | |
| | pentane | benzene | toluene |
| Boiling point of diluent (° C.) | 71.8 | 80.1 | 110.6 |
| Top pressure(kg/cm²G) | 0.5 | 0.5 | 0.5 |
| Condensation Temperature (° C.) | 55.2 | 54.3 | 54.1 |
| D/F | 0.227 | 0.222 | 0.210 |
| Reflux ratio | 15.74 | 6.11 | 3.66 |
| Bottom temperature (° C.) | 96.7 | 103.5 | 136.7 |
| Temperatures from 2^{nd} | 58.6-90.6 | 56.9-99.0 | 56.4-129.4 |
| to 15^{th} plates (° C.) | | | |
| Column diameter (m) | 2.28 | 1.43 | 1.24 |
| CPD reaction loss in column 40 | −787 | −624 | −849 |
| (kg/hr) | | | |
| Recovery of CPD in | 80.55 | 84.48 | 79.26 |
| the overhead (%) | | | |
| Recovery of diluent in | 2.98 | 0.42 | 0.00 |
| the overhead (%) | | | |

With reference to Tables 8A to 9B, the diluent with a boiling point higher than that of MCPD (72.8° C.), such as benzene and toluene, is concentrated in the lower section of column 40 and almost all discharged from line 19. In this case, the upper section of column 40 has a high CPD concentration to promote the formation of DCPD, such that a great amount of DCPD is formed and discharged from the bottom of column 40; thus reducing the recovery of CPD at the overhead of column 40.

TABLE 9A
Flow rates and compositions of the overhead stream (line 18)
and the bottom stream (line 19) of column 40 in Example 5.

| | | Diluent | | | |
|---|---|---|---|---|---|
| | | | cyclo- | 2-methyl | |
| | | n-pentane | pentane | pentane | n-hexane |
| Line 18 | Flow rate | 12637 | 12549 | 10601 | 4670 |
| | (kg/hr) | | | | |
| | CPD (wt %) | 32.85 | 33.24 | 39.29 | 79.57 |
| | MCPD (wt %) | 0.06 | 0.06 | 0.07 | 0.16 |
| | Diluent (wt %) | 67.09 | 66.70 | 60.64 | 20.27 |
| Line 19 | Flow rate | 3004 | 3093 | 5041 | 10971 |
| | (kg/hr) | | | | |
| | CPD (wt %) | 0.22 | 0.21 | 0.13 | 0.06 |
| | MCPD (wt %) | 1.12 | 1.75 | 4.48 | 4.04 |
| | DCPD (wt %) | 2.89 | 3.25 | 1.70 | 2.89 |
| | MDCPD | 5.89 | 3.41 | 0.45 | 0.21 |
| | (wt %) | | | | |
| | TCPD (wt %) | 0.12 | 0.08 | 0.03 | 0.01 |
| | Diluent (wt %) | 0.12 | 3.60 | 41.75 | 70.96 |
| | Other (wt %) | 89.64 | 87.70 | 51.29 | 21.82 |

TABLE 9B
Flow rates and compositions of the overhead stream (line 18)
and the bottom stream (line 19) of column 40 in Example 5.

| | | Diluent | | |
|---|---|---|---|---|
| | | Methyl | | |
| | | cyclo- | | |
| | | pentane | Benzene | Toluene |
| Line 18 | Flow rate (kg/hr) | 3556 | 3480 | 3283 |
| | CPD (wt %) | 92.48 | 98.73 | 99.77 |
| | MCPD (wt %) | 0.21 | 0.21 | 0.22 |
| | Diluent (wt %) | 7.30 | 1.05 | 0.00 |
| Line 19 | Flow rate (kg/hr) | 12086 | 12162 | 12358 |
| | CPD (wt %) | 0.05 | 0.05 | 0.05 |
| | MCPD (wt %) | 3.32 | 3.33 | 1.29 |
| | DCPD (wt %) | 6.31 | 4.82 | 5.99 |
| | MDCPD (wt %) | 0.47 | 0.71 | 1.97 |
| | TCPD (wt %) | 0.01 | 0.01 | 0.02 |
| | Diluent (wt %) | 69.85 | 71.42 | 70.67 |
| | Other (wt %) | 19.98 | 19.65 | 20.02 |

The boiling points of n-hexane, methyl cyclopentane, and MCPD are quite close to each other. As shown in Tables 8A to 9B, with n-hexane and methyl cyclopentane as the diluent, the reflux ratio of column 40 has to be high enough to prevent MCPD from moving forward to the overhead of column 40 with the diluent. Therefore, the column diameter and the liquid holdup on each plate of column 40 have to be increased, leading to more DCPD formation and loss of CPD recovery in the overhead of column 40.

Other diluents with boiling points between CPD and MCPD, such as 2-methyl pentane and cyclopentane, are also included in Example 5. With reference to Tables 8A and 8B, the CPD concentration in liquid phase is more diluted with these diluents in the upper section of column 40; thereby moderating the formation of DCPD and increasing the recovery of CPD in the overhead of column 40.

As shown in Table 8A, when n-pentane was taken as the diluent, it is completely distilled from the overhead of column 40, since its boiling point is lower than that of CPD. In this case, bottom temperature of column 40 tends to increase to cause more dimerization of CPD in the reboiler; therefore, the recovery of CPD in the overhead is reduced. On the other hand, the dimerization of CPD in upper section of column 40 tends to reduce due to high concentration of n-pentane, which will compensate the loss of CPD in the reboiler.

Accordingly, n-pentane, cyclopentane, 2-methyl pentane, and n-hexane are the preferred diluents for high CPD recovery.

### EXAMPLE 6

### Dimerization of Cyclopentadiene and Recovery of Diluent

As statement preceding, the intention of mixing the second blend in line 18 with the recycled diluent via line 25 in FIG. 1, is to form the combined stream having a lower CPD concentration in line 21than the second blend. The CPD concentration of the combined stream in line 21 is designated to be about 30 wt % based on the weight of the combined stream in line 21. The combined stream in line 21 is fed to the first dimerization reactor 50. Reactor 50 is simulated with the tubular reactor model of ASPEN 14 operated under adiabatic mode and at the pressure of 9.9 kg/cm² G. The volume of reactor 50 is set at 16.493 m³. The effluent of reactor 50 discharged through line 22 as the fourth blend, is fed to the 5^{th} plate of the first recovery column 60 to recover the diluent by the overhead stream of column 60 via line 23 while to obtain a high purity dicyclopentadiene comprising a concentration of DCPD more than 95 wt % from the bottom of column 60 via line 24. The recovered diluent in line 23 is recycled through lines 25, 31 and 33.

Column 60 is operated under a top pressure of −0.6 kg/cm² G and a reflux ratio of 0.5. Due to the decomposition and synthesis of dimers also occur in column 60, operation of column 60 is simulated with Aspen Plus under reactive distillation mode.

Besides the method of adding diluent to reduce CPD concentration in line 18, the temperature of reactor 50 is also set to control the production of CPD trimer in the adiabatic dimerization reactor. Therefore, the inlet temperature of reactor 50 in line 21 is regulated to let the maximum allowable temperature at the outlet of reactor 50 set at 120° C.

The simulated operating parameters, calculated CPD conversion, the flow rates and compositions at the inlet (line 21) and the outlet (line 22) of the first dimerization reactor 50 with four preferred diluents, including n-pentane, cyclopentane, 2-methyl pentane and n-hexane, which are screened in Example 5, are shown in Table 10.

For producing a high purity DCPD product in the bottom of column 60 while recovering the most of the diluent in the feed to column 60, which is the fourth blend, by the overhead stream of column 60, the minimum recovery of the diluent in the feed to column 60 by the overhead stream is 99.95% of the feed to the column 60. In the calculation by Aspen Plus, distillate-to-feed mass ratio (D/F) of column 60 is adjusted to meet the above requirement. Four preferred diluents, including n-pentane, cyclopentane, 2-methyl pentane, and n-hexane, are used to illustrate in the present example.

The simulated operating parameters, calculated loss of DCPD decomposition, the flow rates and compositions of the overhead stream in line 23 and the bottom stream in line 24 for column 60 with the four diluents mentioned above are shown in Table 11.

TABLE 10
Operating parameters, calculated CPD conversion, simulation
results, flow rate and composition at the inlet (line
21) and outlet (line 22) of reactor 50 in Example 6

| | | Diluent | | | |
|---|---|---|---|---|---|
| | | | cyclo- | 2-methyl | |
| | | n-pentane | pentane | pentane | n-hexane |
| Pressure (kg/cm²G) | | 9.90 | 9.90 | 9.90 | 9.90 |
| Inlet temperature (° C.) | | 74.7 | 63.4 | 71.3 | 70.0 |
| Residence time (min) | | 41.9 | 49.7 | 43.0 | 46.7 |
| Outlet temperature (° C.) | | 119.3 | 118.9 | 119.3 | 119.3 |
| CPD conversion (%) | | 90.1 | 90.5 | 89.9 | 90.8 |
| Line 21 | Flow rate | 14032 | 14125 | 14442 | 13571 |
| | (kg/hr) | | | | |
| | CPD (wt %) | 29.98 | 29.98 | 29.98 | 29.97 |
| | MCPD (wt %) | 0.05 | 0.05 | 0.06 | 0.08 |
| | Diluent (wt %) | 69.96 | 69.96 | 69.96 | 69.94 |
| Line 22 | Flow rate | 14032 | 14125 | 14442 | 13571 |
| | (kg/hr) | | | | |
| | CPD (wt %) | 2.98 | 2.83 | 3.03 | 2.75 |
| | MCPD (wt %) | 0.02 | 0.02 | 0.02 | 0.03 |
| | DCPD (wt %) | 26.86 | 27.01 | 26.81 | 27.07 |
| | MDCPD | 0.06 | 0.06 | 0.07 | 0.09 |
| | (wt %) | | | | |
| | TCPD (wt %) | 0.12 | 0.12 | 0.11 | 0.12 |
| | Diluent (wt %) | 69.96 | 69.96 | 69.96 | 69.94 |

TABLE 11
Operating parameter, simulation results, and flow rates
and compositions of the overhead stream (line 23) and
the bottom stream (line 24) of column 60 in Example 6

| | | Diluent | | | |
|---|---|---|---|---|---|
| | | | Cyclo- | 2-methyl | |
| | | n-pentane | pentane | pentane | n-hexane |
| Top pressure | | −0.6 | −0.6 | −0.6 | −0.6 |
| (kg/cm²G) | | | | | |
| Condensation | | 12.1 | 24.4 | 33.5 | 40.4 |
| Temperature (° C.) | | | | | |
| D/F | | 0.729 | 0.729 | 0.731 | 0.728 |
| Reflux ratio | | 0.50 | 0.50 | 0.50 | 0.50 |
| Bottom temperature | | 143.3 | 148.9 | 149.2 | 149.6 |
| (° C.) | | | | | |
| Column diameter (m) | | 1.44 | 1.42 | 1.44 | 1.41 |
| Net loss of DCPD | | −5.78 | −19.08 | −16.59 | −18.47 |
| decomposition (kg/hr) | | | | | |
| Line 23 | Flow rate | 10223 | 10297 | 10555 | 9882 |
| | (kg/hr) | | | | |
| | CPD (wt %) | 4.01 | 4.06 | 4.29 | 3.95 |
| | MCPD (wt %) | 0.00 | 0.01 | 0.03 | 0.04 |
| | Diluent (wt %) | 95.99 | 95.92 | 95.68 | 96.00 |
| Line 24 | Flow rate | 3809 | 3827 | 3888 | 3689 |
| | (kg/hr) | | | | |
| | CPD (wt %) | 0.22 | 0.01 | 0.01 | 0.01 |
| | MCPD (wt %) | 0.05 | 0.03 | 0.01 | 0.00 |
| | DCPD (wt %) | 98.80 | 99.16 | 99.18 | 99.06 |
| | MDCPD | 0.24 | 0.23 | 0.24 | 0.34 |
| | (wt %) | | | | |
| | DMDCPD | 0.00 | 0.01 | 0.00 | 0.00 |
| | (wt %) | | | | |
| | TCPD (wt %) | 0.56 | 0.44 | 0.43 | 0.46 |
| | Diluent (wt %) | 0.13 | 0.13 | 0.13 | 0.13 |

### EXAMPLE 7

### Recovery of Diluent in Third Blend

According to Table 9A in Example 5, when 2-methyl pentane or n-hexane is selected as diluent, the third blend in line 19 (the bottom stream of the heavy removal column 40) contains a large amount of diluent to be recovered. However, as shown in Table 9A, the third blend in line 19 contains a certain amount of MCPD. If a distillation column is straightforwardly applied to recover diluent from the third blend, the most portion of MCPD goes with the recycled diluent, which will lead to the accumulation o f MCPD in the process and then will reduce the purity and recovery of DCPD. Therefore, when the boiling point of selected diluent is close to that of MCPD, it is very crucial to keep the recovered diluent away from MCPD in the bottom stream of column 40.

The present example demonstrates a method to recover the diluent from the bottom stream of the heavy removal column 40 and to keep its purity high. Referring to FIG. 2, the bottom stream of column 40, which is withdrawn via line 19, is charged to the second dimerization reactor 70 to dimerize MCPD into DMDCPD; The DMDCPD, which has a boiling point much higher than the boiling point of the diluent. Reactor 70 is simulated by the tubular reactor model of ASPEN 14 operated under adiabatic mode and under a pressure of 9.9 kg/cm² G. The volume of reactor 70 is set at 10.387 m³. The effluent of reactor 70 discharged through line 27 is fed to the 9^{th} plate of the second recovery column 80 to recover the diluent in the effluent of reactor 70 by the overhead stream of column 80 that is withdrawn via line 28, and to produce a fuel oil by-product from the bottom of column 80 that is piped through line 29. Column 80 is operated under a top pressure of 0 kg/cm² G. Since the decomposition and synthesis of dimers still occur in column 80, the operation of column 80 is simulated with Aspen Plus under reactive distillation mode.

For the purpose of promoting the dimerization of MCPD in the second dimerization reactor 70, the inlet temperature of reactor 70 is set at 160° C. when taking 2-methyl pentane as the diluent and is set at 170° C. when taking n-hexane as the diluent. The simulated operating parameters, calculated MCPD conversion, and the flow rate and composition at the inlet (line 21) and the outlet (line 22) of reactor 70 with 2-methyl pentane and n-hexane as diluent are shown in Table 12.

In order to obtain a recovered diluent with high purity in the overhead of column 80, the maximum allowable of toluene in the overhead stream (line 28) of column 80 is 2% of the feed to column 80, while the minimum recovery of diluent in the overhead stream (line 28) of column 80 is 99.9% of the feed to column 80. In the simulation calculations, reflux ratio (RR) and distillate-to-feed mass ratio (D/F) of column 80 are adjusted to meet these two requirements.

The simulated operating parameters as well as calculated recovery of diluent in the overhead of column 80 for two series of diluents, which are 2-methyl pentane and n-hexane, are shown in Table 13. The flow rates and compositions of the overhead stream in line 28 and the bottom stream in line 29 for column 80 are given in Table 13 as well.

TABLE 12
Operation parameters, simulation results, flow rate and
composition at the inlet (line 19) and the outlet (line
27) of second dimerization reactor 70 in Example 7

Diluent

| | | 2-methyl pentane | n-hexane |
|---|---|---|---|
| Pressure (kg/cm²G) | | 9.9 | 9.9 |
| Inlet temperature (° C.) | | 160.0 | 170.0 |
| Residence time (min) | | 74.3 | 31.0 |
| Outlet temperature (° C.) | | 170.0 | 177.6 |
| MCPD conversion (%) | | 97.45 | 94.38 |
| Line 19 | Flow rate (kg/hr) | 5041 | 10971 |
| | CPD (wt %) | 0.13 | 0.06 |
| | MCPD (wt %) | 4.48 | 4.04 |
| | Diluent (wt %) | 41.75 | 70.96 |
| Line 27 | Flow rate (kg/hr) | 5041 | 10971 |
| | CPD (wt %) | 0.06 | 0.09 |
| | MCPD (wt %) | 0.11 | 0.23 |
| | DCPD (wt %) | 1.63 | 2.77 |
| | MDCPD (wt %) | 0.74 | 0.39 |
| | DMDCPD (wt %) | 13.75 | 6.28 |
| | TCPD (wt %) | 0.04 | 0.02 |
| | Diluent (wt %) | 41.75 | 70.96 |

TABLE 13
Operation parameters, simulation results, flow rate, and composition
of the overhead stream (line 28) and the bottom stream (line
29) of second recovery column 80 in Example 7

| | | Diluent | |
|---|---|---|---|
| | | 2-methyl pentane | n-hexane |
| Top pressure (kg/cm²G) | | 0.00 | 0.00 |
| Condensation temperature (° C.) | | 60.1 | 68.4 |
| D/F | | 0.42 | 0.71 |
| Reflux ratio | | 1.04 | 1.30 |
| Bottom temperature (° C.) | | 184.9 | 183.3 |
| Column diameter (m) | | 0.63 | 1.30 |
| Diluent recovery at the overhead | | 99.9 | 99.9 |
| stream (%) | | | |
| Line 28 | Flow rate (kg/hr) | 2116 | 7843 |
| | CPD (wt %) | 0.32 | 0.49 |
| | MCPD (wt %) | 0.22 | 0.31 |
| | Diluent (wt %) | 99.35 | 99.17 |
| | Toluene (wt %) | 0.11 | 0.03 |
| Line 29 | Flow rate (kg/hr) | 2925 | 3129 |
| | CPD (wt %) | 0.01 | 0.02 |
| | MCPD (wt %) | 0.03 | 0.01 |
| | DCPD (wt %) | 2.67 | 8.77 |
| | MDCPD (wt %) | 1.28 | 1.38 |
| | DMDCPD (wt %) | 23.70 | 22.02 |
| | TCPD (kg/hr) | 0.06 | 0.08 |
| | Others (wt %) | 72.17 | 67.46 |
| | Diluent (wt %) | 0.07 | 0.25 |

### EXAMPLE 8

### Fabrication of High Purity Dicyclopentadiene

The present example illustrates the method for making high purity DCPD from the bottom stream of a debutanizer, using cyclopentane (CP) as diluent. With reference to FIG. 1, a hydrocarbon originated from the bottom of a debutanizer with the composition shown in Table 1 is fed to the 18^{th} plate of a first distillation column 10 by line 11 at rate of 42000 kg/hr and at temperature of 150° C., to remove C5 and lighter hydrocarbons by the overhead stream withdrawn via line 12. The first distillation column 10 is operated under a top pressure of 1.0 kg/cm² G, a reflux ratio of 3.0 and the distillate-to-feed mass ratio of 0.366. The bottom stream of the distillation column 10 discharged via line 13 is introduced to the 14^{th} plate of a second distillation column 20 via line 13 at temperature 121.3° C. to remove the C6 to C8 hydrocarbons from the top of the second distillation column 20 via line 14. The second distillation column 20 is operated under a top pressure of −0.7336 kg/cm² G, a reflux ratio of 2.0, and a distillate-to-feed mass ratio (D/F) of 0.744. The bottom stream of the second distillation column 20 discharged from line 15 is mixed with a recycled diluent from line 31 and a makeup diluent from line 32 to from a combined stream in line 16. The combined stream in line 16, which is the first blend, is then charged to a cracker 30. The cracker 30 is operated under isothermal mode at 340° C. and a residence time of 0.5 second. The effluent of the cracker 30 is fed to the 11^{th} plate of a heavy removal column 40 via lines 35 and 17, to recover CPD and diluent in the effluent of the cracker 30 (the feed of the heavy removal column 40, or the cracked feedstock) by the overhead stream of the heavy removal column 40 via line 18, and to remove the remaining hydrocarbons in the effluent of the cracker 30 by the bottom stream of the heavy removal column 40 via line 19. The heavy removal column 40 is operated under a top pressure of 0.5 kg/cm² G, a reflux ratio of 1.30 and a distillate-to-feed mass ratio of 0.80. The overhead stream of the column 40 withdrawn via line 18 is mixed with a recycled diluent from line 25 to form a combined stream in line 21.The combined stream in line 21 is fed to a first dimerization reactor 50 under a temperature of 64.3° C. The first dimerization reactor 50 is operated under adiabatic mode, at a pressure of 9.9 kg/cm² G, and with a residence time of 49.7 minutes. The effluent of the first dimerization reactor 50 withdrawn via line 22, which is the fourth blend, is then fed to the 5^{th} plate of a first recovery column 60 to recover the diluent in the fourth blend by the overhead stream of the first recovery column 60 withdrawn via line 23, while to produce a product comprising DCPD from the bottom of the first recovery column 60. The product comprising DCPD is discharged via line 24. The first recovery column 60 is operated under a top pressure of −0.6 kg/cm² G, a reflux ratio of 0.5 and a distillate-to-feed mass ratio of 0.73.

The flow rates and compositions of streams mentioned in Example 8 are shown in Table 14. The product comprising DCPD discharged via line 24 is a high purity DCPD comprising a concentration of DCPD of 99.16 wt %.

TABLE 14
Flow rates and compositions of streams mentioned in Example 8

| | | Composition | | | | | | |
|---|---|---|---|---|---|---|---|---|
| Line | Flow rate | CPD | CP | MCPD | DCPD | MDCPD | TCDP | Other |
| No. | (kg/hr) | (wt %) | (wt %) | (wt %) | (wt %) | (wt %) | (wt %) | (wt %) |
| 11 | 42000 | 9.67 | 0.45 | 1.10 | 7.97 | 2.15 | 0.00 | 78.65 |
| 12 | 15373 | 24.31 | 1.24 | 0.00 | 0.00 | 0.00 | 0.00 | 74.45 |
| 14 | 19822 | 0.53 | 0.00 | 1.49 | 0.00 | 0.00 | 0.00 | 97.98 |
| 15 | 6805 | 0.01 | 0.00 | 0.00 | 52.29 | 13.45 | 0.02 | 34.23 |
| 16 | 15642 | 2.26 | 54.22 | 0.01 | 22.75 | 5.85 | 0.01 | 14.89 |
| 19 | 3093 | 0.21 | 3.60 | 1.75 | 3.25 | 3.41 | 0.08 | 87.70 |
| 23 | 10297 | 4.06 | 95.92 | 0.01 | 0.00 | 0.00 | 0.00 | 0.00 |
| 24 | 3827 | 0.01 | 0.13 | 0.03 | 99.16 | 0.23 | 0.44 | 0.01 |
| 25 | 1576 | 4.05 | 95.94 | 0.01 | 0.00 | 0.00 | 0.00 | 0.00 |
| 31 | 8721 | 4.05 | 95.94 | 0.01 | 0.00 | 0.00 | 0.00 | 0.00 |
| 32 | 115 | 0.00 | 100.00 | 0.00 | 0.00 | 0.00 | 0.00 | 0.00 |

Based on the above description, the advantages of the method for making high purity dicyclopentadiene in accordance with the present invention are summarized as follows.

1. By selecting the saturated C5 hydrocarbon, the saturated C6 hydrocarbon, benzene or the mixture thereof as the diluent for the dimer stock, the first blend with a lower concentration of DCPD and MDCPD than the dimer stock is obtained. By introducing the first blend, instead of the dimer stock, to the cracker for the vapor phase cracking, the clogging problem of cracker tubes is avoided.

2. The diluent of the present invention is separated from the high purity dicyclopentadiene and then recycled back to the process and reused. Therefore, the cost of diluent is low.

3. The diluent used in the present invention is not only to solve the clogging problem of the dimer cracker but also to reduce the concentration of dienes in liquid phase and the temperature of the distillation column. Those can moderate the CPD dimerization in the distillation column. Therefore, the recovery of DCPD of the hydrocarbon stream originated from the bottom of the debutanizer is increased and the yield of the high purity dicyclopentadiene is ensured.

4. By the diluent of the present invention, as the adiabatic dimerization of cyclopentadiene is processed, the diluent reduces both the reaction rate of CPD dimerization and the amount of heat generated in a unit time, which can prevent the excessive temperature rise in reactor and minimize the trimerization of cyclopentadiene. The production of the high purity dicyclopentadiene is ensured.

Even though numerous characteristics and advantages of the present invention have been set forth in the foregoing description, together with details of the structure and features of the invention, the disclosure is illustrative only. Changes may be made in the details, especially in matters of shape, size, and arrangement of parts within the principles of the invention to the full extent indicated by the broad general meaning of the terms in which the appended claims are expressed.

What is claimed is:

1. A method for making high purity dicyclopentadiene comprising steps of:
providing a dimer feedstock comprising dicyclopentadiene and methyl dicyclopentadiene;
blending the dimer feedstock and a diluent selected from a group consisting of: a saturated C5 hydrocarbon, a saturated C6 hydrocarbon, benzene and a mixture thereof, to obtain a first blend;
cracking the first blend in a vapor phase at a cracking temperature ranging from 300° C. to 400° C. to obtain a cracked feedstock comprising cyclopentadiene, methyl cyclopentadiene and the diluent;
separating the cracked feedstock into a second blend and a third blend, the second blend comprising cyclopentadiene and the diluent, and the third blend comprising methyl cyclopentadiene and the diluent;
heating the second blend to a dimerizing temperature ranging from 50° C. to 120° C. to obtain a fourth blend comprising dicyclopentadiene and the diluent; and
separating the fourth blend into a recycled diluent and a high purity dicyclopentadiene having a concentration of dicyclopentadiene greater than 95 weight percent based on the weight of the high purity dicyclopentadiene;
wherein the diluent comprises the recycled diluent.

2. The method as claimed in claim 1, wherein the method further comprises a step of:
separating the recycled diluent into a first recycled diluent and a second recycled diluent;
the step of blending the dimer feedstock and the diluent to obtain the first blend comprises:
blending the dimer feedstock and the first recycled diluent to obtain the first blend; and
the step of cracking the first blend in the vapor phase at the cracking temperature ranging from 300° C. to 400° C. to obtain the cracked feedstock comprises
cracking the first blend in the vapor phase at the cracking temperature ranging from 300° C. to 400° C. into an intermediate product; and
blending the intermediate product with the second recycled diluent to obtain the cracked feedstock.

3. The method as claimed in claim 1, wherein the method further comprises a step of:
separating the recycled diluent into a first recycled diluent and a third recycled diluent;
the step of blending the dimer feedstock and the diluent to obtain the first blend comprises
blending the dimer feedstock and the first recycled diluent to obtain the first blend; and
the step of heating the second blend to the dimerizing temperature ranging from 50° C. to 120° C. to obtain the fourth blend comprises
blending the second blend and the third recycled diluent and then heating to the dimerizing temperature ranging from 50° C. to 120° C. to obtain the fourth blend.

4. The method as claimed in claim 2, wherein the step of separating the recycled diluent into the first recycled diluent and the second recycled diluent comprises
separating the recycled diluent into the first recycled diluent, the second recycled diluent and a third recycled diluent; and
the step of heating the second blend to the dimerizing temperature ranging from 50° C. to 120° C. to obtain the fourth blend comprises
blending the second blend and the third recycled diluent and then heating to the dimerizing temperature ranging from 50° C. to 120° C. to obtain the fourth blend.

5. The method as claimed in claim 1, wherein the method further comprises steps of:
heating the third blend to dimerize the methyl cyclopentadiene of the third blend and obtain a high-boiling point hydrocarbon feedstock; and
separating the high-boiling point hydrocarbon feedstock to obtain a high-boiling point hydrocarbon and a fourth recycled diluent; and
the step of blending the dimer feedstock and the diluent to obtain the first blend comprises
blending the dimer feedstock and the fourth recycled diluent to obtain the first blend.

6. The method as claimed in claim 2, wherein the method further comprises steps of:
heating the third blend to dimerize the methyl cyclopentadiene of the third blend and obtain a high-boiling point hydrocarbon feedstock; and
separating the high-boiling point hydrocarbon feedstock to obtain a high-boiling point hydrocarbon and a fourth recycled diluent; and
the step of blending the dimer feedstock and the diluent to obtain the first blend comprises
blending the dimer feedstock, the first recycled diluent and the fourth recycled diluent to obtain the first blend.

7. The method as claimed in claim 3, wherein the method further comprises steps of:
heating the third blend to dimerize the methyl cyclopentadiene of the third blend and obtain a high-boiling point hydrocarbon feedstock; and
separating the high-boiling point hydrocarbon feedstock to obtain a high-boiling point hydrocarbon and a fourth recycled diluent; and
the step of blending the dimer feedstock and the diluent to obtain the first blend comprises
blending the dimer feedstock, the first recycled diluent and the fourth recycled diluent to obtain the first blend.

8. The method as claimed in claim 4, wherein the method further comprises steps of:
heating the third blend to dimerize the methyl cyclopentadiene of the third blend and obtain a high-boiling point hydrocarbon feedstock; and
separating the high-boiling point hydrocarbon feedstock to obtain a high-boiling point hydrocarbon and a fourth recycled diluent; and
the step of blending the dimer feedstock and the diluent to obtain the first blend comprises
blending the dimer feedstock, the first recycled diluent and the fourth recycled diluent to obtain the first blend.

9. The method as claimed in claim 1, wherein the saturated C5 hydrocarbon is selected from a group consisting of cyclopentane, n-pentane and isopentane.

10. The method as claimed in claim 8, wherein the saturated C5 hydrocarbon is selected from a group consisting of cyclopentane, n-pentane, and isopentane.

11. The method as claimed in claim 1, wherein the saturated C6 hydrocarbon is selected from a group consisting of cyclohexane, n-hexane, an isomer of cyclohexane and an isomer of n-hexane.

12. The method as claimed in claim 8, wherein the saturated C6 hydrocarbon is selected from a group consisting of cyclohexane, n-hexane, an isomer of cyclohexane and an isomer of n-hexane.

13. The method as claimed in claim 1, wherein in the step of providing the dimer feedstock comprising dicyclopentadiene and methyl dicyclopentadiene, the dimer feedstock comprises a total concentration of dicyclopentadiene and methyl dicyclopentadiene ranging from 50 weight percent to 85 weight percent based on the weight of the dimer feedstock.

14. The method as claimed in claim 8, wherein in the step of providing the dimer feedstock comprising dicyclopentadiene and methyl dicyclopentadiene, the dimer feedstock comprises a total concentration of dicyclopentadiene and methyl dicyclopentadiene ranging from 50 weight percent to 85 weight percent based on the weight of the dimer feedstock.

15. The method as claimed in claim 1, wherein in the step of blending the dimer feedstock and the diluent selected from the group consisting of the saturated C5 hydrocarbon, the saturated C6 hydrocarbon, benzene and the mixture thereof to obtain the first blend, the first blend comprises a total concentration of dicyclopentadiene and methyl dicyclopentadiene ranging from 10 weight percent to 50 weight percent based on the weight of the first blend.

16. The method as claimed in claim 8, wherein in the step of blending the dimer feedstock and the diluent selected from the group consisting of the saturated C5 hydrocarbon, the saturated C6 hydrocarbon, benzene and the mixture thereof to obtain the first blend, the first blend comprises a total concentration of dicyclopentadiene and methyl dicyclopentadiene ranging from 10 weight percent to 50 weight percent based on the weight of the first blend.

17. The method as claimed in claim 1, wherein the step of providing the dimer feedstock comprises
separating a hydrocarbon stream originated from a debutanizer into a hydrocarbon feedstock comprising hydrocarbons with five carbons and another hydrocarbon feedstock comprising hydrocarbons with six or more carbons; and
separating the hydrocarbon feedstock comprising hydrocarbons with six or more carbons to obtain the dimer feedstock.

18. The method as claimed in claim 17, wherein the step of providing the dimer feedstock comprises
separating the hydrocarbon stream originated from the debutanizer comprising a concentration of dicyclopentadiene ranging from five weight percent to 20 weight percent and a concentration of methyl dicyclopentadiene ranging from 0.5 weight percent to 5 weight percent based on the weight of the hydrocarbon stream into the hydrocarbon feedstock comprising hydrocarbons with five carbons and the hydrocarbon feedstock comprising hydrocarbons with six or more carbons; and
separating the hydrocarbon feedstock comprising hydrocarbons with six or more carbons to obtain the dimer feedstock.

19. The method as claimed in claim 8, wherein the step of providing the dimer feedstock comprises
separating a hydrocarbon stream originated from a debutanizer into a hydrocarbon feedstock comprising hydrocarbons with five carbons and another hydrocarbon feedstock comprising hydrocarbons with six or more carbons; and
separating the hydrocarbon feedstock comprising hydrocarbons with six or more carbons to obtain the dimer feedstock.

20. The method as claimed in claim 19, wherein the step of providing the dimer feedstock comprises
separating the hydrocarbon stream originated from the debutanizer comprising a concentration of dicyclopentadiene ranging from 5 weight percent to 20 weight percent and a concentration of methyl dicyclopentadiene ranging from 0.5 weight percent to 5 weight percent based on the weight of the hydrocarbon stream into the hydrocarbon feedstock comprising hydrocarbons with five carbons and the hydrocarbon feedstock comprising hydrocarbons with six or more carbons; and
separating the hydrocarbon feedstock comprising hydrocarbons with six or more carbons to obtain the dimer feedstock.
